(19) United States
(12) Patent Application Publication
(10) Pub. No.: US 2013/0047963 A1
(43) Pub. Date: Feb. 28, 2013

(54) WARRANTY VIOLATION DETECTION SYSTEM FOR DISALLOWED FUELS

(71) Applicant: John J. Horsting, Williamsburg, VA (US)

(72) Inventor: John J. Horsting, Williamsburg, VA (US)

(73) Assignee: CONTINENTAL AUTOMOTIVE SYSTEMS US, INC., Deer Park, IL (US)

(21) Appl. No.: 13/218,543

(22) Filed: Aug. 26, 2011

(51) Int. Cl. F02M 69/46; G01N 33/00

(52) U.S. Cl. 123/456; 73/61.41

(57) ABSTRACT

A fuel supply system (10) for a vehicle includes a fuel tank (12) for holding fuel therein. A pump (14) pumps fuel from the tank. A filter (16) filters fuel. A fuel rail (20) receives filtered fuel originating from the pump. A pressure regulator (18) controls pressure of fuel received by the fuel rail. Fuel injectors (24) inject fuel, received from the fuel rail, into an internal combustion engine. A fuel detection system (28) in in the fuel supply system and includes a plurality of detectors (30, 30′). Each detector is constructed and arranged to be exposed to fuel to detect a distinct chemical element or compound in the fuel.

### FIELD OF THE INVENTION

The invention relates to fuel systems of internal combustion engines for vehicles and, more particularly, a method and system to detect unwanted and unwarranted chemical compounds in consumer loaded fuels.

### BACKGROUND OF THE INVENTION

Currently, there is no system in place, other than related impacted fault codes in the Engine Condition Monitoring (ECM) as required with the on-board diagnostic (OBD) systems, to note prior presence of adverse chemical compounds within the fuel system of a vehicle. Including the retrospective attempt to forensically demonstrate from causal testing or analysis of any deposits on components, it is very difficult for suppliers or the original equipment manufacturer (OEM) as a warranty provider to the retail customer, to prove a “conditions of use” violation that relieves the supplier and/or OEM of any warranty repair cost.

Proof of unauthorized substances in fuel are today derived from forensic testing on the components involved, typically long after the repairs have been done on the consumer's vehicle or after the OEM relation with the supplier has been substantially degraded. There is substantial lag time for such forensic testing and once completed, there is no definitive proof that the customer used a disallowed fuel.

Thus, there is a need to detect unwarranted chemical compounds in consumer loaded fuels via methods that are obvious and visual to dealer service personnel in a tamper resistant manner.

### SUMMARY OF THE INVENTION

An object of the invention is to fulfill the need referred to above. In accordance with the principles of the present invention, this objective is obtained by providing a fuel supply system for a vehicle including a fuel tank for holding fuel therein, the fuel tank having a filler neck; a pump for pumping fuel from the tank; a filter for filtering fuel; a fuel rail for receiving filtered fuel originating from the pump; a pressure regulator for controlling pressure of fuel received by the fuel rail; and fuel injectors for injecting fuel, received from the fuel rail, into an internal combustion engine. The filler neck, tank, pump, filter, pressure regulator, and fuel rail are fluidly connected to define fuel flow path structure between the tank and the fuel injectors. A fuel detection system is in fluid communication with the fuel flow path structure. The fuel detection system includes a plurality of detectors. Each detector is constructed and arranged to be exposed to fuel to detect a distinct chemical element or compound in the fuel.

In accordance with another aspect of a disclosed embodiment, a method of detecting distinct chemical elements or compounds in fuel in a fuel supply system provides a fuel supply system of a vehicle to supply fuel from a fuel tank to a fuel rail, with the fuel rail feeding fuel injectors with fuel. The method provides a fuel detection system in the fuel supply system so as to be exposed to fuel to detect distinct chemical elements or compounds in the fuel.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, wherein like reference numerals refer to like parts, in which:

FIG. 1 illustrates a fuel supply system according to an embodiment including a fuel detection system according to an embodiment.

### DETAILED DESCRIPTION OF THE EXEMPLARY EMBODIMENTS

The embodiment provides a system and method to detect unwanted and unwarranted chemical compounds in consumer loaded fuels. The system protects the OEM and/or supplier from consumer attempts to disguise major engine damage caused by use of unauthorized fuel.

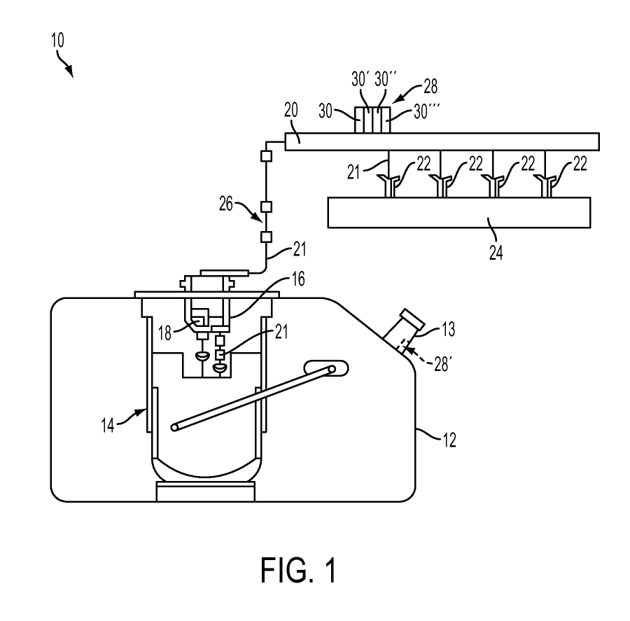

FIG. 1 illustrates a fuel supply system, generally indicated at 10, including a tank 12 having a filler neck 13, a pump 14, a filter 16, a pressure regulator 18, a fuel rail 20, and fuel injectors 22 received fuel from the fuel rail 20 for injecting fuel into an internal combustion engine 24. The tank 12 holds fuel and the pump 14 is shown connected to an inside of the fuel tank 12. The filter 16 and the pressure regulator 18 are shown connected inside the pump 14. However, the filter 16 and the pressure regulator 18, either individually or as an integral combination, can be connected on the exterior of the pump 14, or can be connected remotely with respect to the pump 14 so long as filtered fuel under regulated pressure is delivered to the fuel rail 20. The filler neck 13, tank 12, pump 14, filter 16, pressure regulator 18, fuel rail 20 are fluidly connected (such as by piping 21) to define fuel flow path structure 26 between the tank 12 and the fuel injectors 22. The pressure regulator 18 can be coupled to a tap in piping between the pump 14 and the filter 16, or between the filter 16 and the fuel rail 20 to control pressure of fuel that is received by the fuel rail 20. Fuel that is bled-off by the pressure regulator 18 is returned to the pump 14 or tank 12. The filtered fuel supplied to the fuel rail 20 by the pump 14 is supplied to each of the injector(s) 22, and subsequently supplied to the engine 24.

In accordance with an embodiment, a fuel detection system is shown, generally indicated at 28, in fluid communication with the fuel flow path structure 26. In the embodiment, the fuel detection system 28 is shown fluidly coupled with the fuel rail 20 for ease of access thereto. However, the fuel detection system 28 can be coupled anywhere within the fuel flow path structure 26 such as on the fuel pump 14, the filter 16, the regulator 18, so long that it is exposed to fuel. The fuel detection system 28′ can be provided in the filler neck 13, with a transparent window alerting the operator to a fueling violation, especially for fleet vehicles.

The fuel detection system 28 includes a plurality of chemical element or chemical compound detectors 30, 30′, 30″, 30′″, etc. that are exposed to fuel to detect parts per million (ppm) of specific chemical elements or compounds in the fuel. Compounds of interest that have existing test methods available include, but are not limited to, ethanol, methanol, water, sulfides, etc. These compounds impact the life of the fuel injectors, and other fuel system components, by low lubricity, aggressive attack, corrosion, and deposit formation. Thus, for example, detector 30 can be configured to detect ethanol, detector 30′ can be configured to detect methanol, detector 30″ can be configured to detect water or water vapor, and detector 30′″ can be configured to detect sulfides. The detectors 30, 30′, 30″, 30′″ can be in the form of detector tubes manufactured by Gastec Corporation of Fukaya, Ayase-City, Japan (such as Model No. 6, No. 111L, No. 112L). Alternatively, the detectors 30, 30′, 30″, 30′″ can be dehydrogenase-based biosensors, bioassay tests, strips, or chemical-optical kits. To detect sulfide, a conventional sulfide-selective optode membrane can be used.

Such detectors offer the possibility of permanent change, not erasable by subsequent lack of continued exposure, or by human intervention short of replacing the entire detection system 28. The detectors 30, 30′, 30″, 30′″ can detect varying levels of ppm of distinct chemical compounds/elements.

The increasing interest in bio fuels, typically utilized in given and limited concentrations within conventional gasoline or diesel fuel, as well as emerging country markets, leave open the real and documented possibilities of consumers utilizing higher percentages of bio additives in fuels than what their vehicles are certified to. This may also occur without the consumer's awareness at unbranded, low control, retail fueling stations. The belief that government regulations protect the integrity of the retail fuel supply ignores proven violations in developing countries, as well as the developed countries. There is potential of consumers using brewed bio fuels, as well as the fact that vehicles remain unprotected from their owner's misuse of fuels since there is no dedicated fueling nozzle configuration per fuel composition (excluding leaded vs. non-leaded fuel, since separate nozzle configurations are provided for these fuels). While OBDs might indicate a fault light if the offending chemical imbalance is detected, these codes are typically suggestive and not definitive, as well as erasable by elimination of 12 V power to the ECM. Further, the reliance of most OBD systems on inductance, conductivity, or capacitance, ignores the possibility of changes, or lack thereof, in these parameters caused by varying fuel components or contaminants that may have markedly differing impacts on the operation or material of supplied device components used in the fuel systems. An example of this is identical pH leading to identical conductivity readings of given concentrations of acetic acid, vs. hydrochloric acid, vs. sulfuric acid. These three acids differ markedly in their attack on metals, and are documented to exist in varying quantities in both bio and regular fuels from various countries, depending on the local fuel processing used.

A given diffusion rate of the tested fuel stream across the reactive media of the detector 30 may be needed. Such cases would require a labyrinth or orifice built within fuel rail 20, regulator 18 or other supplied fuel system components in order to achieve the required diffusion rate.

The fuel detection system 28 can be an integral member in the fuel flow path structure 26 such as being integral with the fuel rail 20 as noted above, or can be an add-on member. Thus, an aftermarket add-on detection system 28 is possible in a package design that can be retrofitted to any vehicle, or applied as a new OEM accessory. Packaging of multiple detectors 30, each specific to a given chemical compound, can also be accomplished in a single unit by modular slotting, similar to either health care industry blood or other specimen testing or personal computer assembly where purchase specified requirements can be simply added via modules into a series of available slots or cubes. In this manner, even though a given vehicle may be produced in a single country, its use might become country specific, and a vehicle selected for export might be given a different detector set than a domestic unit.

The fuel detection system 28 or 28′ provides an easy and effective way for vehicle service personnel to determine if improper fuel was used by the vehicle's operator and thus, determine if the vehicle warranty was violated. The service personnel could merely access the detection system 28 or 28′ and inspect the detectors 30, 30′, etc., for the presence of any chemical element or compound detected that should not be present in the fuel system. The detection system 28 or 28′ is completely passive, with no sampling, active analysis, processing, electrical current, etc. required.

With the system 28, 28′, there is no data loss with the loss of 12 V power, and there is no data loss with fuel change over, post any incident. Further, depending on the customer, country, and economic cycles, circumstances may easily result in fuel aging in a given vehicle. This aging period is easily measured in weeks, which is the same time span that a given batch of fuel exists in the United States prior to complete consumption of that market batch. Still further, corrections to low pH in fuel stocks, while easily accomplished by sodium bicarbonate, result in dramatically increased fuel conductivity, with resultant galvanic corrosion of fuel system components. As a result, an insult to fuel system components may be due to attempts to mitigate the primary deficiency of the fuel. This system of multiple detectors enhances the ability to identify possible vehicle owner interaction with the vehicle fuel, and/or adulteration by the retail fuel dealer, by logging multiple insults. Further, by adding a “dead end” spur, with an additional set of multiple detectors, to the fuel line via a “T” fitting, it is also possible to calculate the approximate timing of multiple insults by means of the probable diffusion time of the insult through the distance of the static fuel in the length of the spur line. Likewise there are embodiments and variations possible by use of multiple detector sets and tie- ins to the vehicle ECM to perform additional functions, such as driver notification via the instrument panel, dealer notification of OBD fault codes, termination of engine function to avoid further damage, identification of subsequent insult events by means of alternative parallel fuel paths with detector sets, etc. In the case of General Motor's Onstar, it would also be possible to download fuel information to the Onstar center. It is noted that embodiments that include visibility of the data to other than dealer maintenance personnel are contrary to a tamper proof benefit, but these can be constructed in such a manner, should it be desirable, to include hurdles to tampering, or the benefits of visibility may be seen to out-way the loss of tamper proofing.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A fuel supply system for a vehicle comprising:
a fuel tank for holding fuel therein, the fuel tank having a filler neck,
a pump for pumping fuel from the tank,
a filter for filtering fuel,
a fuel rail for receiving filtered fuel originating from the pump,
a pressure regulator for controlling pressure of fuel received by the fuel rail,
fuel injectors for injecting fuel, received from the fuel rail, into an internal combustion engine, the filler neck, tank, pump, filter, pressure regulator, and fuel rail are fluidly connected to define fuel flow path structure between the tank and the fuel injectors, and
a fuel detection system in fluid communication with the fuel flow path structure, the fuel detection system including a plurality of detectors, each detector being constructed and arranged to be exposed to fuel to detect a distinct chemical element or compound in the fuel.

2. The fuel supply system of claim 1, wherein a detector is constructed and arranged to detect ethanol.

3. The fuel supply system of claim 1, wherein a detector is constructed and arranged to detect methanol.

4. The fuel supply system of claim 1, wherein a detector is constructed and arranged to detect water.

5. The fuel supply system of claim 1, wherein a detector is constructed and arranged to detect sulfides.

6. The fuel supply system of claim 5, wherein the detector includes a sulfide-selective optode membrane.

7. The fuel supply system of claim 1, wherein a first detector is constructed and arranged to detect ethanol, a second detector is constructed and arranged to detect methanol, and a third detector is constructed and arranged to detect water.

8. The fuel supply system of claim 7, wherein each of the first, second, and third detectors is a detector tube.

9. The fuel supply system of claim 1, wherein the fuel detection system is fluidly coupled with the fuel rail.

10. The fuel supply system of claim 1, wherein the fuel detection system is disposed within the filler neck.

11. A fuel supply system for a vehicle comprising:
a fuel tank for holding fuel therein, the fuel tank having a filler neck,
means for pumping fuel from the tank,
means for filtering fuel,
a fuel rail for receiving filtered fuel originating from the pump,
means for controlling pressure of fuel received by the fuel rail,
fuel injectors for injecting fuel, received from the fuel rail, into an internal combustion engine, the filler neck, tank, means for pumping, means for filtering, means for controlling, and the fuel rail are fluidly connected to define fuel flow path structure between the tank and the fuel injectors, and
means, in fluid communication with the fuel flow path structure, for detecting distinct chemical elements or compounds in the fuel.

12. The fuel supply system of claim 11, wherein the means for detecting includes a plurality of detectors, each detector being constructed and arranged to be exposed to fuel to detect a distinct chemical element or compound in the fuel.

13. The fuel supply system of claim 12, wherein a detector is constructed and arranged to detect sulfides.

14. The fuel supply system of claim 13, wherein the detector includes a sulfide-selective optode membrane.

15. The fuel supply system of claim 11, wherein a first detector is constructed and arranged to detect ethanol, a second detector is constructed and arranged to detect methanol, and a third detector is constructed and arranged to detect water.

16. The fuel supply system of claim 15, wherein each of the first, second, and third detectors is a detector tube.

17. The fuel supply system of claim 11, wherein the means for detecting is fluidly coupled with the fuel rail.

18. The fuel supply system of claim 11, wherein the means for detecting is disposed within the filler neck.

19. A method of detecting distinct chemical elements or compounds in fuel in a fuel supply system, the method comprising:
providing a fuel supply system of a vehicle to supply fuel from a fuel tank to a fuel rail, with the fuel rail feeding fuel injectors with fuel, and
providing a fuel detection system in the fuel supply system so as to be exposed to fuel to detect distinct chemical elements or compounds in the fuel.

20. The method of claim 19, further comprising:
inspecting elements or compounds detected by the fuel compound detection system to determine if improper fuel was used in the fuel supply system.
